(19) United States
(12) Patent Application Publication
(10) Pub. No.: US 2023/0038646 A1
(43) Pub. Date: Feb. 9, 2023

(54) NOVEL AND POTENT JAK/STAT INHIBITOR

(71) Applicant: Texas Tech University System, Lubbock, TX (US)

(72) Inventors: Sanjay K. Srivastava, Abilene, TX (US); Itishree Kaushik, Framingham, MA (US)

(21) Appl. No.: 17/793,434

(22) PCT Filed: Jan. 20, 2021
(86) PCT No.: PCT/US2021/014081

§ 371 Date: Jul. 18, 2022

Related U.S. Application Data
(60) Provisional application No. 62/963,258, filed on Jan. 20, 2020.

(51) Int. Cl. A61K 31/35

(52) U.S. Cl. CPC A61K 31/35

(57) ABSTRACT

The present invention includes a method of inhibiting or reducing deregulated JAK tyrosine kinase activity or JAK/ STAT signaling in a subject with a disease by administering Moxidectin or derivatives thereof in an amount sufficient to treat the disease in a subject, a therapeutically or prophylactically effective amount of the compound of Formula I or pharmaceutically acceptable salt thereof.

### CROSS-REFERENCE TO RELATED APPLICATIONS

This application claims priority to U.S. Provisional Application Serial No. 62/963,258, filed Jan. 20, 2020, the entire contents of which are incorporated herein by reference.

### TECHNICAL FIELD OF THE INVENTION

The present invention relates to methods of reducing or inhibiting cancer in a subject, and the use of derivatives, compositions, analogs, salts, formulations and structural modification that include Moxidectin for preventing or treating disorder (s) related to JAK kinase and its downstream effector molecules such as STAT’s (a transcription factor).

### STATEMENT OF FEDERALLY FUNDED RESEARCH

None.

### INCORPORATION-BY-REFERENCE OF MATERIALS FILED ON COMPACT DISC

None.

### BACKGROUND OF THE INVENTION

Without limiting the scope of the invention, its background is described in connection with protein kinases.

Protein kinases are enzymes that chemically modify other proteins by catalyzing the transfer of gamma phosphates from nucleotide triphosphates, often adenosine triphosphate (ATP), and covalently attaching them to a free hydroxyl group of amino acid residues serine, threonine and tyrosine.

Approximately 30% of all human proteins may be modified by kinase activity. Protein kinases direct the enzymatic activity, cellular location and primary function/association of substrate proteins and regulate cell signal transduction and cell function coordination.

Research studies have shown that aberrant expression of normal or mutated protein kinases are frequently associated with the formation and propagation of a number of diseases. Studies have shown that overexpression or inappropriate protein kinase expression is associated with cancer, cardiovascular disease, rheumatoid arthritis, diabetes, ocular disease, neurologic disorders and autoimmune disease. Thus, investigating compounds that potently inhibit the activity and function of protein kinases will allow for a greater understanding of the physiological roles of protein kinases.

The Janus kinase 2 (JAK2) kinase gene is a non-receptor tyrosine kinase that signals through by members of the type II cytokine receptor family. Examples of type II cytokine receptors include interferon receptors, gp130 receptors (e.g., IL-6R), GM-CSF receptors (e.g., IL-3R, IL-5R and GM-CSF-R), and single chain receptors (e.g., Epo-R, Tpo-R, GH-R, PRL-R). In humans, JAK2 is on chromosome 9 and is encoded by the JAK2 gene which is Entrez Gene ID: 3717, and protein sequence UniProtKB - 060674 (JAK2_HUMAN).

JAK2 has up to seven JAK homology domains (JH1-JH7) and differs from other JAK kinases by the lack of Src homology binding domains (SH2/SH3). Nonetheless the terminal JH domains retain a high level of homology to tyrosine kinase domains. The carboxy-terminal JH domain (JH1) retains full kinase function while JH2 has reduced kinase activity.

### SUMMARY OF THE INVENTION

The present invention includes a method of inhibiting or reducing deregulated JAK tyrosine kinase activity, or its downstream transcription factor STAT and/or deregulated expression in a subject with a disease which comprises administering to the subject having or suspected to have the disease, a therapeutically or prophylactically effective amount of Moxidectin of Formula I:

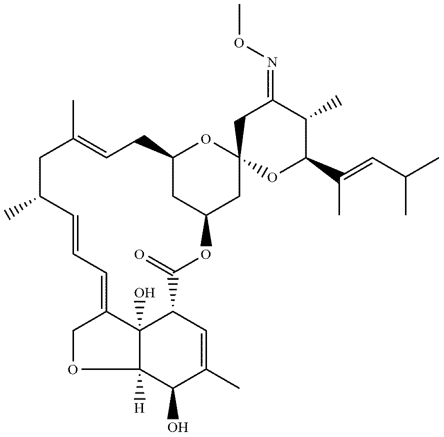 [SMILES: CO/N=C1\C[C@]2(C[C@@H]3C[C@@H](C/C=C(\C)C[C@@H](C)/C=C/C=C4\CO[C@@H]5[C@H](O)C(C)=C[C@@H](C(=O)O3)[C@]45O)O2)O[C@H](/C(C)=C/C(C)C)[C@H]1C]

or a pharmaceutically acceptable carrier, salt or solvate, chemical modification, formulations, analogs, polymorphs thereof. In one aspect, the disease is selected from at least one of: brain, medulloblastoma, neuroblastoma, glioblastoma, leukemia, myeloma, myeloproliferative disease, myelodysplastic syndrome, idiopathic hypereosinophilic syndrome (HES), bladder cancer, breast cancer, cervical cancer, CNS cancer, colon cancer, esophageal cancer, head and neck cancer, liver cancer, lung cancer, nasopharyngeal cancer, neuroendocrine cancer, ovarian cancer, pancreatic cancer, prostate cancer, renal cancer, salivary gland cancer, small cell lung cancer, skin cancer, stomach cancer, testicular cancer, thyroid cancer, uterine cancer, or hematologic malignancy. In one aspect, the disease is selected from any other medical condition with deregulated JAK/STAT signaling selected from at least one of: Polycythemia vera (PV), idiopathic hypereosinophilic syndrome (HES), Severe combined immunodeficiency (SCID), Hyperimmunoglobulin E syndrome (HIES, or Job’s syndrome), Mucocutaneous candidiasis, Mycobacterial infection, or Autoimmune disorders. In another aspect, the therapeutically and prophylactically effective amounts are from about 0.1 to 25, 1 to 15, 2 to 10, 3 to 8, 5 to 15, 2, 3, 4, 5, 6, 7, 8, 9, 10, 12, 15, 20, 25, 30, 35, 40, 45, 50, 55, 60, 65, 70, 75, 80, 85, 90, or 100 mg per day. In another aspect, the compound is administered at least one of continuously, intermittently, systemically, or locally. In another aspect, the deregulated JAK kinase is defined further as a mutated JAK kinase is constitutively active. In another aspect, Moxidectin, active polymorphs, analogs or salts thereof is adapted for oral, enteral, parenteral, intravenous, pulmonary, nasal, intraperitoneal, ocular, topical, otic, intraosseous, nasal, vaginal, intrathecal, sublingual, buccal, respiratory, (nasal), transdermal, or intramuscular administration. In another aspect, the therapeutically or prophylactically effective amount of Moxidectin is administered daily for as long as the subject is in need of treatment for the disease. In another aspect, the composition is provided at least one of sequentially or concomitantly, with another pharmaceutical agent such as chemotherapeutic agents, immunotherapeutic agents Moxidectin’s analog, salt selected from at least one of a phosphate, sulphate, nitrate, di phosphate, bicarbonate, carbonate, clavulanate, isothionate, borate, halide, nitrate, acetate, succinate, lactate, lactobionate, laurate, mandelate, malate, citrate, fumarate, maleate, oleate, oxalate, ascorbate, nicotinate, benzoate, mesylate, salicylate, stearate, tannate, tosylate, valerate, methanesulfonate, ethanesulfonate, benzenesulfonate, ptoluensulfonate, 2-ethane disulfonate, tartrate or naphthalenesulfonate, modified structure or formulations of any kind in a newly diagnosed disease subject, to maintain remission, or a relapsed/refractory disease subject. In another aspect, Moxidectin is provided as a single agent or in combination with another pharmaceutical agent in a newly diagnosed disease subject, to maintain remission, or a relapsed/refractory disease subject. In another aspect, Moxidectin is provided as a single agent or in combination with another pharmaceutical agent, chemotherapeutic agents, immunemodulatory agent, antibodies, miRNA’s in a newly diagnosed disease pediatric subject, to maintain remission, or a relapsed/refractory disease pediatric subject. In another aspect, the subject is relapsed/refractory to prior JAK/STAT signaling pathway. In another aspect, the further comprises the step of determining if the subject is relapsed/refractory to a prior JAK2/STAT signaling pathway inhibitor prior to providing the subject with treatment.

In another embodiment, the present invention includes a method for treating a subject with a disease comprising: administering to the subject in need of such treatment a therapeutically effective amount of Moxidectin or a salt thereof, wherein the disorder is characterized by deregulated JAK/STAT signaling. Proliferative disease is selected from at least one of a brain, medulloblastoma, neuroblastoma, glioblastoma, leukemia, myeloma, myeloproliferative disease, myelodysplastic syndrome, idiopathic hypereosinophilic syndrome (HES), bladder cancer, breast cancer, cervical cancer, CNS cancer, colon cancer, esophageal cancer, head and neck cancer, liver cancer, lung cancer, nasopharyngeal cancer, neuroendocrine cancer, ovarian cancer, pancreatic cancer, prostate cancer, renal cancer, salivary gland cancer, small cell lung cancer, skin cancer, stomach cancer, testicular cancer, thyroid cancer, uterine cancer, and hematologic malignancy. In one aspect, the compound is administered orally, intravenously, or intraperitoneally. In another aspect, the Moxidectin is provided at least one of sequentially or concomitantly, with chemotherapy, radiotherapy, immunotherapy, miRNA mediated therapy, bone marrow transplant, hormone therapy, targeted drug delivery, cryoblation, radiofrequency ablation, clinical trials or surgery in a newly diagnosed disease, to maintain remission, or a relapsed/refractory proliferative disease. In another aspect, the Moxidectin is provided as a single agent or in combination with chemotherapy, radiotherapy, immunotherapy or surgery for treatment of a pediatric subject with the disease. In another aspect, the Moxidectin is provided as a single agent to at least one of post standard induction therapy, or high dose induction therapy, in newly diagnosed proliferative disease. In another aspect, the Moxidectin is provided as a single agent in treatment of subjects with the disease that is either refractory to, or has relapsed after, standard or high dose chemotherapy, radiotherapy, immunotherapy or surgery. In another aspect, the subject is relapsed/refractory to at least one other tyrosine kinase inhibitor, including but not limited to sorafenib, quizartinib, PLX3397, sunitinib, Midostaurin, or Lestaurtinib.

Yet another embodiment of the present invention includes a method for treating a subject suffering from brain cancer comprising: obtaining a sample from the subject suspected of having a brain cancer; determining from the subject sample that the subject has a deregulated JAK/STAT signaling; and administering to the subject in need of such treatment a therapeutically effective amount of Moxidectin, a salt form, polymorphs, analogs, formulations, modified structures thereof, wherein the brain cancer is characterized by deregulated JAK/STAT signaling.

Another embodiment of the present invention includes a method for specifically inhibiting a deregulated receptor tyrosine kinase comprising: obtaining a subject sample and determining which receptor tyrosine kinases are deregulated; and administering to a mammal in need of such treatment a therapeutically effective amount of Moxidectin or a salt thereof, wherein the deregulated signaling is JAK/STAT signaling. In one aspect, the proliferative disease is selected from at least one of a brain, medulloblastoma, neuroblastoma, glioblastoma, leukemia, myeloma, myeloproliferative disease, myelodysplastic syndrome, idiopathic hypereosinophilic syndrome (HES), bladder cancer, breast cancer, cervical cancer, CNS cancer, colon cancer, esophageal cancer, head and neck cancer, liver cancer, lung cancer, nasopharyngeal cancer, neuroendocrine cancer, ovarian cancer, pancreatic cancer, prostate cancer, renal cancer, salivary gland cancer, small cell lung cancer, skin cancer, stomach cancer, testicular cancer, thyroid cancer, uterine cancer, and hematologic malignancy. In another aspect, the therapeutically and prophylactically effective amounts are from about 0.1 to 25, 1 to 15, 2 to 10, 3 to 8, 5 to 15, 2, 3, 4, 5, 6, 7, 8, 9, 10, 12, 15, 20, 25, 30, 35, 40, 45, 50, 55, 60, 65, 70, 75, 80, 85, 90, or 100 mg per day. In another aspect, the compound is administered at least one of continuously, intermittently, systemically, or locally. In another aspect, the deregulated JAK/STAT signaling is defined further as a mutated JAK kinase is constitutively active. In another aspect, Moxidectin is administered through oral, enteral, parenteral, intravenous, pulmonary, nasal, intraperitoneal, ocular, topical, otic, intraosseous, nasal, vaginal, intrathecal, sublingual, buccal, respiratory, (nasal), transdermal, or intramuscular routes of administration. In another aspect, the therapeutically or prophylactically effective amount of the compound is administered daily for as long as the subject is in need of treatment for the disease. In one aspect, the subject is provided treatment, one or more subject samples are obtained to determine the effect of the treatment, and treatment is continued until the disease is reduced or eliminated. In another aspect, Moxidectin is provided at least one of sequentially or concomitantly, with another pharmaceutical agent, chemotherapeutic agent, immune modulatory agent or antibodies in a newly diagnosed disease subject, to maintain remission of an existing subject, or a relapsed/refractory disease subject. In another aspect, the present invention is provided as a single agent or in combination with another pharmaceutical agent in a newly diagnosed disease subject, to maintain remission, or a relapsed/refractory disease subject. In another aspect, the present invention is provided as a single agent or in combination with another pharmaceutical agent in a newly diagnosed disease pediatric or adult subject, to maintain remission, or a relapsed/refractory proliferative disease pediatric subject. In another aspect, the subject is relapsed/refractory to a prior JAK/STAT signaling inhibitor.

Yet another embodiment of the present invention includes a method for treating a subject with cancer comprising: obtaining a sample suspected of having cancer from the subject; determining if the subject that has become resistant to prior JAK inhibition; and administering a therapeutically effective amount of Moxidectin, its salt form, analog, formulations thereof to overcome the resistance to the prior JAK protein tyrosine kinase inhibition.

The present invention provides methods of reducing or inhibiting the kinase activity of JAK/STAT signaling in a cell or a subject, and the use of such methods for preventing or treating cell associated disorder (s) related to JAK/STAT signaling. Other features and advantages of the invention will be apparent from the following detailed description of the invention and from the claims.

### BRIEF DESCRIPTION OF THE DRAWINGS

For a more complete understanding of the features and advantages of the present invention, reference is now made to the detailed description of the invention along with the accompanying figure in which:

FIGS. 1A to 1C show that Moxidectin treatment suppresses the proliferation of brain cancer cells. FIG. 1A shows the treatment of Daoy (medulloblastoma), FIG. 1B of IMR32 (neuroblastoma), and FIG. 1C of KNS42 (glioblastoma) cells with varying concentrations of moxidectin displayed significant reduction in the survival of cells in a concentration and time-dependent manner (FIGS. 1A-1C).

FIGS. 2A to 2C show that induction of apoptosis by Moxidectin treatment. FIG. 2A shows the treatment of Daoy (medulloblastoma), FIG. 2B of KNS42 (glioblastoma), and FIG. 2C of IMR32 (neuroblastoma) cells with varying concentrations of moxidectin. The percentage of apoptotic cells with 20 µM moxidectin treatment ranged from 90-100% in all the brain cancer cell lines tested (FIGS. 2A-2C).

FIG. 3 shows that Moxidectin inhibits JAK/STAT signaling. Daoy, IMR32, and KNS42 cells were treated with 0, 10, 15 and 20 µM moxidectin for 48 hours and evaluated by western blotting.

FIGS. 4A to 4D show that Moxidectin suppresses the growth of subcutaneously implanted brain cancer cells. These results show that 10 mg/kg moxidectin suppressed the growth of subcutaneously injected medulloblastoma cells by 90% (FIG. 4A). Similarly, 2.5 mg/kg and 5 mg/kg moxidectin suppressed Daoy tumor growth by 45% and 60% respectively (FIG. 4B). FIG. 4C shows that 2.5 mg/kg and 5 mg/kg moxidectin suppressed Daoy tumor growth by 45% and 60% respectively. FIG. 4D shows that Moxidectin was able to inhibit -65% of intracranially implanted medulloblastoma tumors in vivo.

FIGS. 5A-5I are graphs that shows the effects of Moxidectin treatment suppresses the proliferation of brain cancer cells: Treatment of Daoy, UW228, ONS76, PFSK1 (medulloblastoma), IMR32, SK-N-AS (neuroblastoma) and SF268, SF295 (glioblastoma) cells with varying concentrations of moxidectin displayed significant reduction in the survival of cells in a concentration and time-dependent manner (FIGS. 5A-5I, respectively).

FIGS. 6A-6F shows the induction of apoptosis by moxidectin: The mode of cell death caused by moxidectin treatment was determined by Annexin V/FITC assay in Daoy, UW228, ONS76, UW426, PFSK1 and IMR32 brain cancer cells (FIGS. 6A-6F, resepectively).

FIG. 7 shows that Moxidectin inhibits JAK-STAT signaling: shows that moxidectin mediates its anti-cancer effects in brain cancer cell lines, Daoy, IMR32, and KNS42 cells were treated with 0, 10, 15 and 20 µM moxidectin for 48 hours and evaluated by western blotting.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

The present invention comprises the use of the compounds of the present invention to inhibit JAK/STAT signaling in a cell or a subject, or to treat disorders related to JAK/STAT signaling activity or expression in a subject.

In one embodiment to this aspect, the present invention provides a method for reducing or inhibiting the kinase activity of JAK in a cell comprising the step of contacting the cell with a compound of the present invention. The present invention also provides a method for reducing or inhibiting the kinase activity of JAK and therefore inhibiting the activation of transcription factor STAT3 and its downstream molecules in a subject comprising the step of administering a compound of the present invention to the subject. The present invention further provides a method of inhibiting cell proliferation in a cell comprising the step of contacting the cell with a compound of the present invention.

As used herein, the term “subject” refers to an animal, such as a mammal or a human, who has been the object of treatment, observation or experiment.

As used herein, the term “contacting” refers to the addition of Moxidectin or pharmaceutically acceptable salt, polymorphs, analog’s or formulations to cells such that the compound is taken up by the cell.

In other embodiments to this aspect, the present invention provides both prophylactic and therapeutic methods for treating a subject at risk or susceptible to developing a cell proliferative disorder driven by aberrant kinase activity of JAK. In one example, the invention provides methods for preventing a disorder related to JAK, comprising administration of a prophylactically effective amount of a pharmaceutical composition comprising a compound of the present invention in a subject. Administration of prophylactic agent Moxidectin can occur prior to the manifestation of symptoms characteristic of the JAK driven cell disorder, such that a disease or disorder is prevented or, alternatively, delayed in its progression.

As used herein, the term “prophylactically effective amount” refers to an amount of Moxidectin active compound or pharmaceutical salt that inhibits or delays in a subject the onset of a disorder as being sought by a researcher, veterinarian, medical doctor or other clinician.

As used herein, the term “therapeutically effective amount” as used herein, refers to an amount of active compound or pharmaceutical salt that elicits the biological or medicinal response in a subject that is being sought by a researcher, veterinarian, medical doctor or other clinician, which includes alleviation of the symptoms of the disease or disorder being treated.

Methods for determining therapeutically and prophylactically effective doses for pharmaceutical compositions comprising a compound of the present invention are known in the art.

As used herein, the term “composition” is intended to encompass a product comprising the specified ingredients in the specified amounts, as well as any product which results, directly or indirectly, from combinations of the specified ingredients in the specified amounts.

As used herein, the terms “disorder related to JAK kinase ,” or “disorders related to JAK receptor,” or “disorders related to JAK receptor tyrosine kinase,” or “JAK/STAT driven cell proliferative disorder” includes diseases associated with or implicating JAK activity, for example, mutations leading to constitutive activation of JAK kinase or aberrant upregulation of JAK/STAT signaling. Examples of “disorders related to JAK” include disorders resulting from over stimulation of JAK due to mutations in JAK, or disorders resulting from abnormally high amount of JAK activity due to abnormally high amount of mutations in JAK. It is known that over-activity of JAK has been implicated in the pathogenesis of many diseases, including the following listed cell proliferative disorders, neoplastic disorders and cancers.

The term “cell proliferative disorders” refers to excess cell proliferation of one or more subset of cells in a multicellular organism resulting in harm (i.e. discomfort or decreased life expectancy) to the multicellular organism. Cell proliferative disorders can occur in different types of animals and humans. As used herein, “cell proliferative disorders” include neoplastic disorders.

The term “neoplastic disorder’ as used herein, refers to a tumor resulting from abnormal or uncontrolled cellular growth. Examples of neoplastic disorders include, but are not limited to the following disorders, for instance: brain, medulloblastoma, neuroblastoma, or glioblastoma.

Examples of myeloproliferative disorders, include: thrombocytopenia, essential thrombocytosis (ET), agnogenic myeloid metaplasia, myelofibrosis (MF), myelofibrosis with myeloid metaplasia (MMM), chronic idiopathic myelofibrosis (UIMF), and polycythemia vera (PV), the cytopenias, and pre-malignant myelodysplastic syndromes; cancers such as glioma cancers, lung cancers, breast cancers, colorectal cancers, prostate cancers, gastric cancers, esophageal cancers, colon cancers, pancreatic cancers, ovarian cancers, and hematological malignancies, including myelodysplasia, multiple myeloma, leukemias, and lymphomas. Examples of hematological malignancies include, for instance, leukemias, lymphomas, Hodgkin’s disease, and myeloma. Also, acute lymphocytic leukemia (ALL), acute myeloid leukemia (AML), acute promyelocytic leukemia (APL), chronic lymphocytic leukemia (CLL), chronic myeloid leukemia (CML), chronic neutrophilic leukemia (CNL), acute undifferentiated leukemia (AUL), anaplastic large-cell lymphoma (ALCL), prolymphocytic leukemia (PML), juvenile myelomonocytic leukemia (JMML), adult T-cell ALL, AML, with trilineage myelodysplasia (AMLITMDS), mixed lineage leukemia (MLL), myelodysplastic syndromes (MDSs), myeloproliferative disorders (MPD), and multiple myeloma (MM).

In a further embodiment, the present invention can be combined with another therapy as a combination therapy for treating or inhibiting the onset of a disorder related to JAK/STAT signaling in a subject. The combination therapy comprises the administration of a prophylactically and therapeutically effective amount of Moxidectin in the present invention and one or more other anti-cell proliferation therapies including, but not limited to, chemotherapy, radiation therapy and immunotherapy.

In an embodiment of the present invention, a compound of the present invention may be administered in combination with chemotherapy. Used herein, chemotherapy refers to a therapy involving a chemotherapeutic agent. A variety of chemotherapeutic agents may be used in combination with the present invention. By way of example only, taxane compounds, specifically docetaxel, is safely administered in combination with a compound of the present invention in a dosage of 75 mg per square meter (mg/m²) of body surface area.

Chemotherapy is known to those skilled in the art. The appropriate dosage and scheme for chemotherapy will be similar to those already employed in clinical therapies wherein the chemotherapy is delivered in combination with other therapies or used alone.

In another embodiment of the present invention, compounds of the present invention may be administered in combination with radiation therapy. Used herein, “radiation therapy” refers to a therapy that comprises the exposure of a subject in need to radiation. Radiation therapy is known to those skilled in the art. The appropriate dosage and scheme for radiation therapy will be similar to those already employed in clinical therapies wherein the radiation therapy is delivered in combination with other therapies or used alone.

In another embodiment of the present invention, the compounds of the present invention may be administered in combination with a targeted therapy. As used herein, “targeted therapy” refers to a therapy targeting a particular class of proteins involved in tumor development or oncogenic signaling. For example, tyrosine kinase inhibitors against vascular endothelial growth factor have been used in treating cancers.

The present invention also includes methods that include the use of a second pharmaceutical agent in addition to compounds of the present invention, the two may be administered simultaneously or sequentially (in either order).

In one embodiment, the present invention therapeutically effective amounts of Moxidectin, which is a compound having formula I:

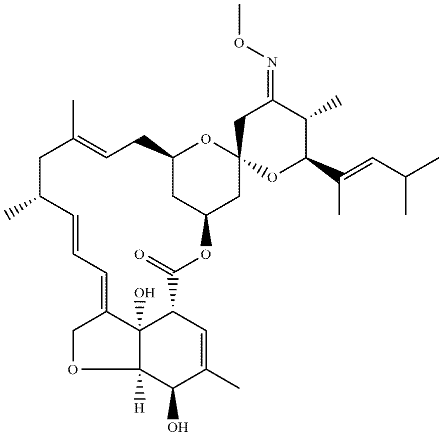 [SMILES: CO/N=C1\C[C@]2(C[C@@H]3C[C@@H](C/C=C(\C)C[C@@H](C)/C=C/C=C4\CO[C@@H]5[C@H](O)C(C)=C[C@@H](C(=O)O3)[C@]45O)O2)O[C@H](/C(C)=C/C(C)C)[C@H]1C]

or a pharmaceutically acceptable salt or solvate, formulations, modified structures, analog’s thereof, in a therapeutically or prophylactically effective amount against a disease is selected from at least one of a brain, medulloblastoma, neuroblastoma, or glioblastoma. Pharmaceutically acceptable salts including phosphate, sulphate, nitrate, di phosphate, bicarbonate, carbonate, clavulanate, isothionate, borate, halide, nitrate, acetate, succinate, lactate, lactobionate, laurate, mandelate, malate, citrate, fumarate, maleate, oleate, oxalate, ascorbate, nicotinate, benzoate, mesylate, salicylate, stearate, tannate, tosylate, valerate, methanesulfonate, ethanesulfonate, benzenesulfonate, ptoluensulfonate, 2-ethane disulfonate, tartrate or naphthalenesulfonate, modified structure or formulations of any kind are prepared in a manner similar to the benzenesulfonate salt and are well known to those of moderate skill in the art.

Moxidectin is an anthelmintic drug used in animals to prevent or control parasitic worms such as heartworm and intestinal worms. Moxidectin is used to treat dogs, cats, horses, cattle and sheep and kills the most common internal and external parasites by selectively binding to a parasite’s glutamate-gated chloride ion channels. These glutamate-gated chloride ion channels are vital to the function of invertebrate nerve and muscle cells. Moxidectin binding leads to the disruption of the glutamate-gated chloride ion channels, thereby disrupting neurotransmission, resulting in that paralysis and death of the parasite. In June 2018, the U.S. Food and Drug Administration approved the use of Moxidectin for the treatment of onchocerciasis in adults and adolescents aged 12 and older.

Moxidectin in the present invention can be administered through oral, enteral, parenteral, intravenous, pulmonary, nasal, intraperitoneal, ocular, topical, otic, intraosseous, nasal, vaginal, intrathecal, sublingual, buccal, respiratory, (nasal), transdermal, or intramuscular routes of administration.

Moxidectin in the present invention may be formulated for slow-release or fast-release with the objective of maintaining contact of compounds of the present invention with targeted tissues for a desired range of time.

Compositions suitable for oral administration include solid forms, such as pills, tablets, caplets, capsules, granules, and powders, liquid forms, such as solutions, emulsions, and suspensions. Forms useful for parenteral administration include sterile solutions, emulsions and suspensions.

The daily dosage of the compounds of the present invention may be varied over a wide range from 0.1 to 25, 1 to 15, 2 to 10, 3 to 8, 5 to 15, 2, 3, 4, 5, 6, 7, 8, 9, 10, 12, 15, 20, 25, 30, 35, 40, 45, 50, 55, 60, 65, 70, 75, 80, 85, 90, or 100 mg per day. The compounds of the present invention may be administered on a daily regimen, once, twice, three or more times per day. Optimal doses to be administered may be determined by those skilled in the art, and will vary with the compound of the present invention used, the mode of administration, the time of administration, the strength of the preparation, the details of the disease condition. One or more factors associated with subject characteristics, such as age, weight, and diet will call for dosage adjustments. Techniques and compositions for making useful dosage forms using the Moxidectin are described in one or more of the following references: Anderson, Philip O.; Knoben, James E.; Troutman, William G, eds., Handbook of Clinical Drug Data, Tenth Edition, McGraw-Hill, 2002; Pratt and Taylor, eds., Principles of Drug Action, Third Edition, Churchill Livingston, New York, 1990; Katzung, ed., Basic and Clinical Pharmacology, Ninth Edition, McGraw Hill, 20037ybg; Goodman and Gilman, eds., The Pharmacological Basis of Therapeutics, Tenth Edition, McGraw Hill, 2001; Remingtons Pharmaceutical Sciences, 20th Ed., Lippincott Williams & Wilkins., 2000; Martindale, The Extra Pharmacopoeia, Thirty-Second Edition (The Pharmaceutical Press, London, 1999); relevant portions incorporated herein by reference.

A dosage unit for use of Moxidectin, may be a single compound or mixtures thereof with other compounds, e.g., a potentiator. The compounds may be mixed together, form ionic or even covalent bonds. The compounds of the present invention may be administered in oral, intravenous (bolus or infusion), intraperitoneal, subcutaneous, or intramuscular form, all using dosage forms well known to those of ordinary skill in the pharmaceutical arts. Depending on the particular location or method of delivery, different dosage forms, e.g., tablets, capsules, pills, powders, granules, elixirs, tinctures, suspensions, syrups, and emulsions may be used to provide the compounds of the present invention to a patient in need of therapy that includes Moxidectin.

The Moxidectin is typically administered in a mixture with suitable pharmaceutical salts, buffers, diluents, extenders, excipients and/or carriers (collectively referred to herein as a pharmaceutically acceptable carrier or carrier materials) selected based on the intended form of administration and as consistent with conventional pharmaceutical practices. Depending on the best location for administration, the Moxidectin may be formulated to provide, e.g., maximum and/or consistent dosing for the particular form for oral, rectal, topical, intravenous injection or parenteral administration. While the Moxidectin may be administered alone, it will generally be provided in a stable salt form mixed with a pharmaceutically acceptable carrier. The carrier may be solid or liquid, depending on the type and/or location of administration selected.

Preparation of the compounds of the present invention. General synthetic methods which may be referred to for preparing the compounds of formula I are provided in U.S. Pat. No. 5,990,146 (issued Nov. 23, 1999) (Warner-Lambert Co.) and PCT published application numbers WO 99/16755 (published Apr. 8, 1999) (Merck & Co.) WO 01/40217 (published Jul. 7, 2001) (Pfizer, Inc.), US Pat. Application Publication No. US 2005/0124599 (Pfizer, Inc.) and U.S. Pat. No. 7,183,414 (Pfizer, Inc.), relevant portions incorporated herein by reference.

In Vitro Assays. The following representative in vitro assays were performed in determining the JAK kinase biological activity of the present invention. These are given to illustrate the invention in a non-limiting fashion.

Inhibition of wild type and mutated JAK kinase activity and specificity for the inhibition of the phosphorylated form of JAK kinase exemplify the specific inhibition of the JAK kinase and cellular processes that are dependent on JAK kinase such as STAT signaling. All of the examples herein show significant and specific inhibition of the JAK kinase and JAK2-dependent cellular responses.

Competitive binding assay. Inhibition of the kinase domain of the human JAK2 receptor was performed using the KINOMEscan KdElect assay protocol. The KINOMEscan platform utilizes a high-throughput competitive binding technology. The assay was performed by combining DNA-tagged kinase, immobilized ligand, and the present invention. The ability of the present invention to compete with immobilized ligand was measured using quantitative PCR of the DNA tag. The competition binding assay was used to evaluate the present invention against a panel of 96 human protein kinases.

Binding constants (Kds) were calculated with a standard dose-response curve using the Hill equation. Curves were fitted using a non-linear least square fit with the Levenberg-Marquardt algorithm. Kds of the present invention were compared to both a negative DMSO control and a positive control compound. The binding affinity of the present invention was visualized using the compound profile visualization interaction map, TREEspot.

### Biological Data for Phosphorylated Kinase Affinity

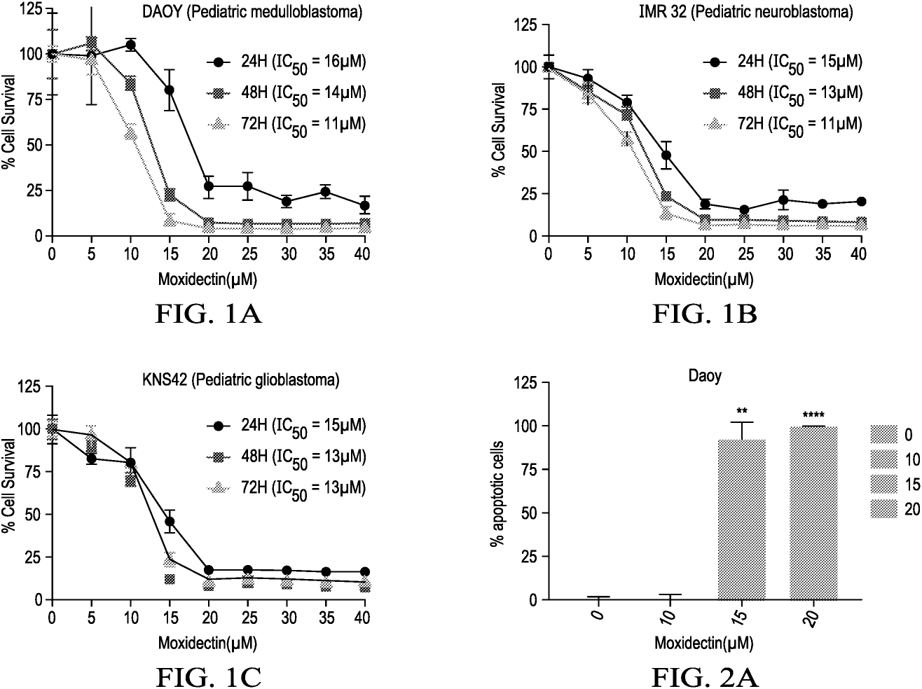

Moxidectin treatment suppresses the proliferation of brain cancer cells. Treatment of Daoy (medulloblastoma), IMR32 (neuroblastoma) and KNS42 (glioblastoma) cells with varying concentrations of moxidectin displayed significant reduction in the survival of cells in a concentration and time-dependent manner (FIGS. 1A-1C). The IC₅₀ of moxidectin was observed to be 11-16 µM in Daoy, IMR32 and KNS42 at 24, 48 and 72-hour time point. These results indicate that moxidectin has the ability to suppress the proliferation of brain cancer cells in a concentration and time-dependent manner.

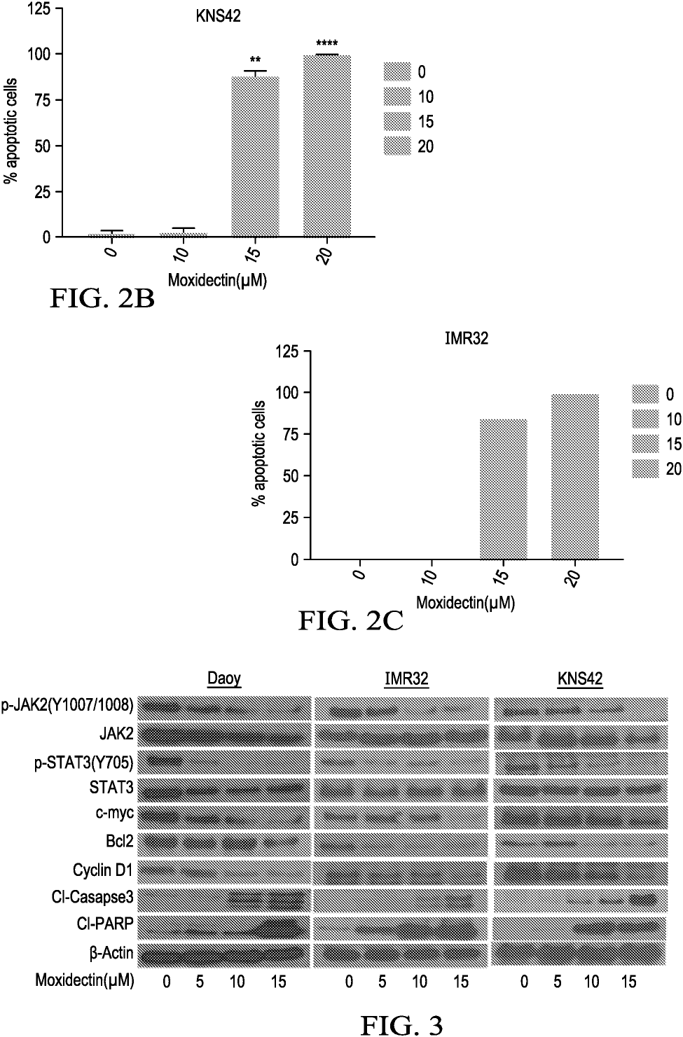

Induction of apoptosis by moxidectin. The mode of cell death caused by moxidectin treatment was determined by Annexin V/FITC assay in Daoy, KNS42 and IMR32 brain cancer cells. As shown in FIGS. 2A-C, treatment with moxidectin for 48 hours resulted in a significant increase of apoptosis in all brain cancer cells. Treatment with 10 µM moxidectin had minimal apoptotic effects on all the three brain cancer cells tested (FIGS. 2A-2C). 15 µM moxidectin induced -80% apoptosis in Daoy, KNS42 and IMR32 brain cancer cell lines. The percentage of apoptotic cells with 20 µM moxidectin treatment ranged from 90-100% in all the brain cancer cell lines tested (FIGS. 2A-2C). Induction of apoptosis by moxidectin treatment in these cell lines was further assessed by increase in the cleavage of caspase 3 and PARP (FIG. 3).

Moxidectin inhibits JAK-STAT signaling. In order to delineate the mechanism by which moxidectin mediates its anti-cancer effects in brain cancer cell lines, Daoy, IMR32, and KNS42 cells were treated with 0, 10, 15 and 20 µM moxidectin for 48 hours and evaluated by western blotting. These results show that moxidectin treatment reduced the phosphorylation of Janus Kinase 2 (JAK2) at Tyr1007/1008 in a concentration-dependent manner. However, protein level of JAK2 remained unchanged. It has been reported that, upon activation (phosphorylation) JAK2 activates multiple downstream transcription factors one of which is STAT3. Hyper activation of STAT3 in cancer cells is strongly attributed to evasion of apoptosis and transcriptional activation of anti-apoptotic proteins such as Bcl-2 and c-myc. These results show that moxidectin treatment reduced the phosphorylation of STAT3 at Tyr705.

Interestingly, it was observed that treatment of brain cancer cells with moxidectin resulted in the reduced expression of anti-apoptotic proteins Bcl-2 in Daoy, IMR32 and KNS42 cell lines (FIG. 3). Moreover, moxidectin increased the cleavage of caspase 3 and PARP as an indication of apoptosis, a primary mode of cell death (FIG. 3). These results suggest that the anti-proliferative and apoptosis inducing effects of moxidectin were mediated by inhibiting JAK2 activation and its downstream targets p-STAT3, c-myc, Bcl2 and Cyclin D1.

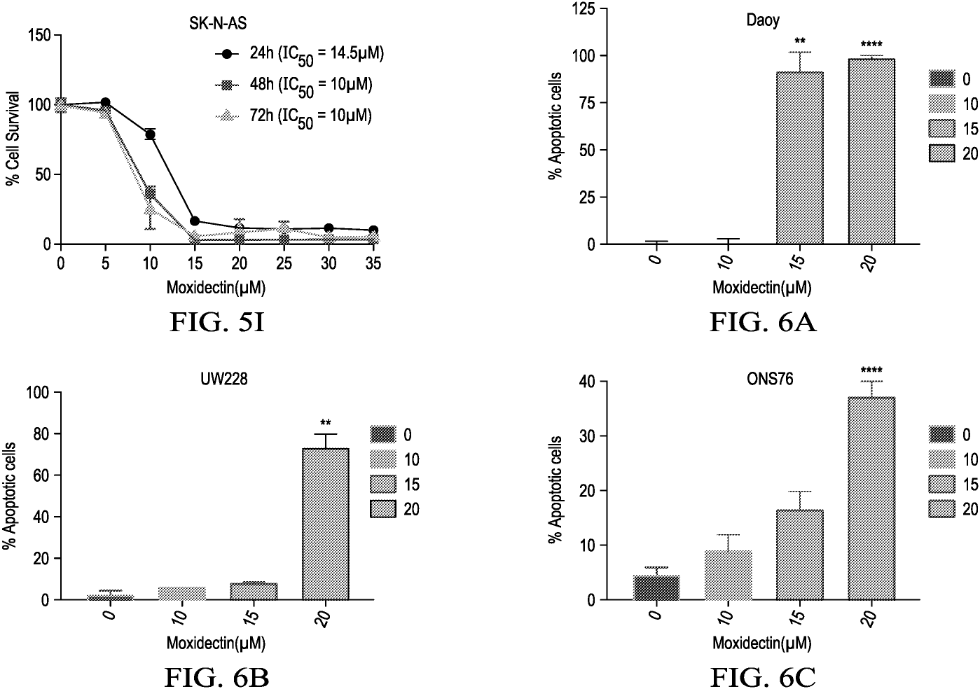

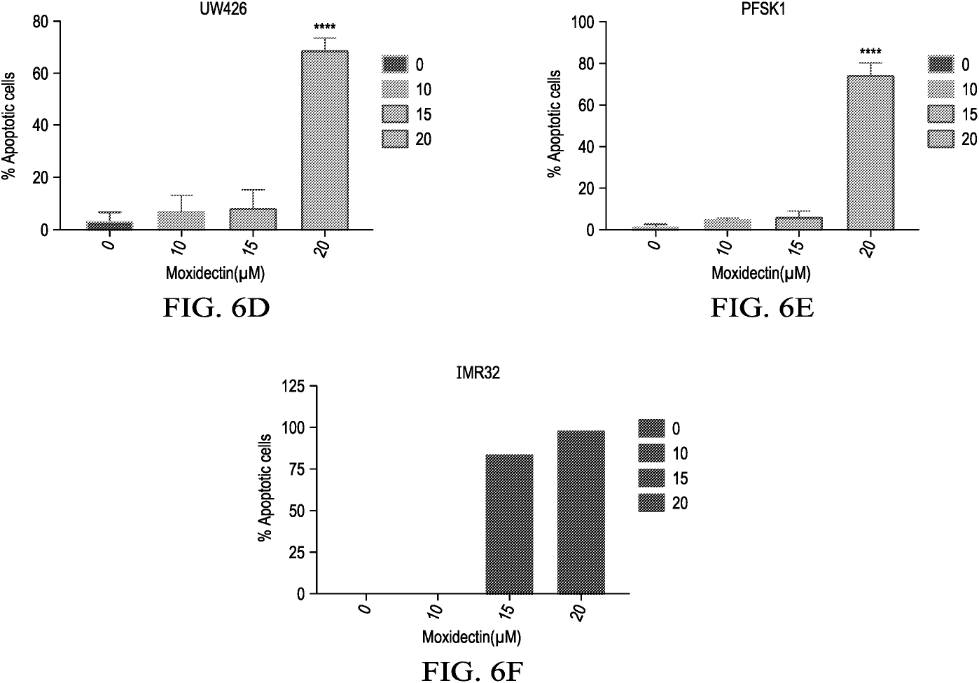

The mode of cell death caused by moxidectin treatment was determined by Annexin V/FITC assay in Daoy, UW228, ONS76, UW426, PFSK1 and IMR32 brain cancer cells (FIGS. 6A-6F, resepectively). As shown in FIGS. 6A-6F, treatment with moxidectin for 48 hours resulted in a significant increase of apoptosis in all brain cancer cells. Treatment with 10 µM moxidectin had minimal apoptotic effects on all the brain cancer cells tested (FIGS. 6A-6F). 15 µM moxidectin induced -80% apoptosis in Daoy and IMR32 brain cancer cell lines. The percentage of apoptotic cells with 20 µM moxidectin treatment ranged from 90-100% in all the brain cancer cell lines. Induction of apoptosis by moxidectin treatment in these cell lines was further assessed by increase in the cleavage of caspase 3 and PARP (FIG. 3).

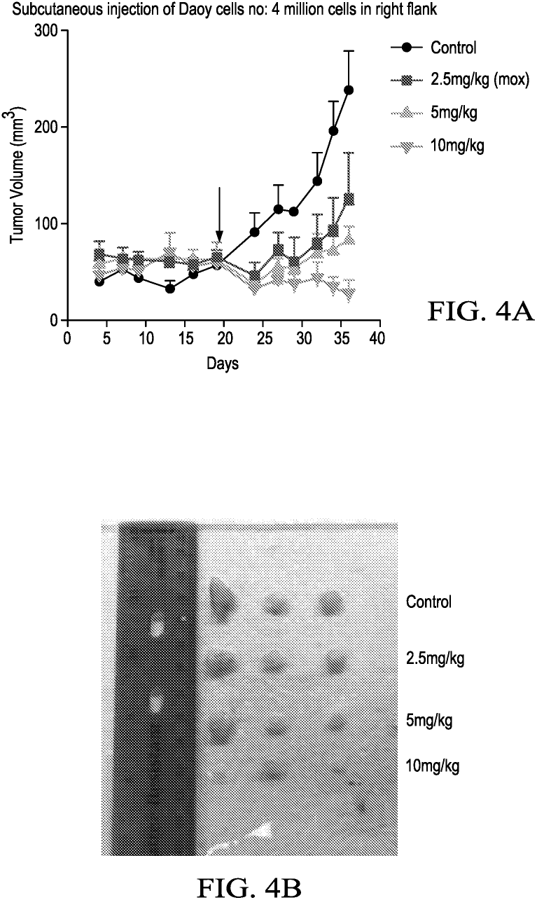

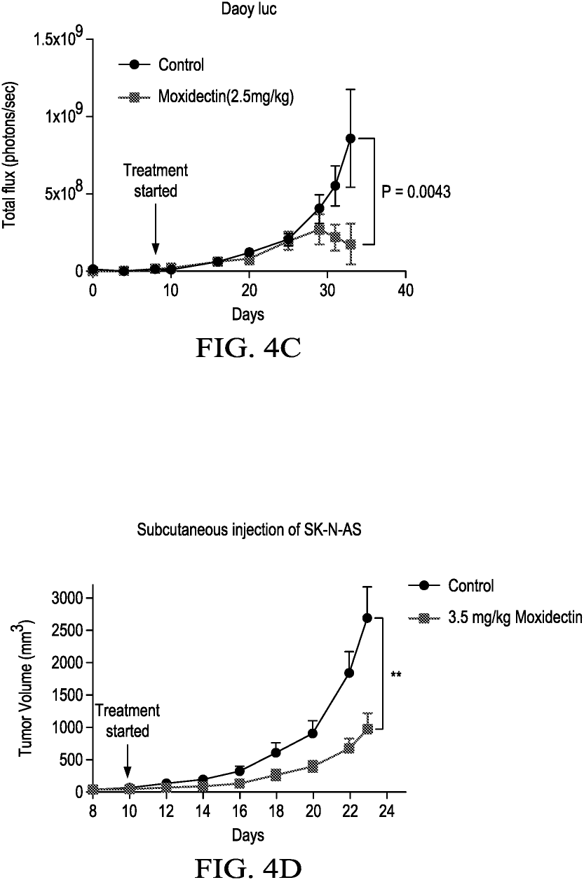

Moxidectin suppresses the growth of subcutaneously implanted brain cancer cells. To evaluate the ability of moxidectin in inhibiting the growth of brain tumor cells in vivo, ~ 4 million Daoy cells were injected subcutaneously into the right flank of the mice. Once the tumor cells started growing and the tumor volume reached ~ 70 mm³, mice were randomized into four groups. Control group received the vehicle whereas the treatment group received 2.5 mg/kg, 5 mg/kg and 10 mg/kg moxidectin once every day. The tumor readings were taken twice a week throughout the course of the experiment using Vernier caliper. These results show that 10 mg/kg moxidectin suppressed the growth of subcutaneously injected medulloblastoma cells by 90% (FIG. 4A). Similarly, 2.5 mg/kg and 5 mg/kg moxidectin suppressed Daoy tumor growth by 45% and 60% respectively (FIG. 4B). These results show that moxidectin inhibits the growth of brain tumor cells in vivo.

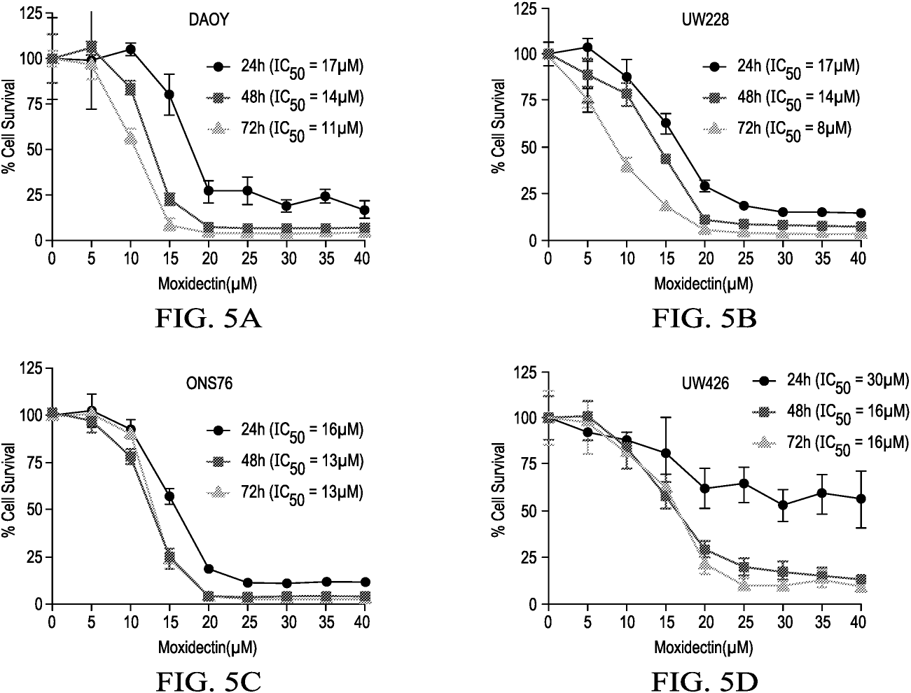

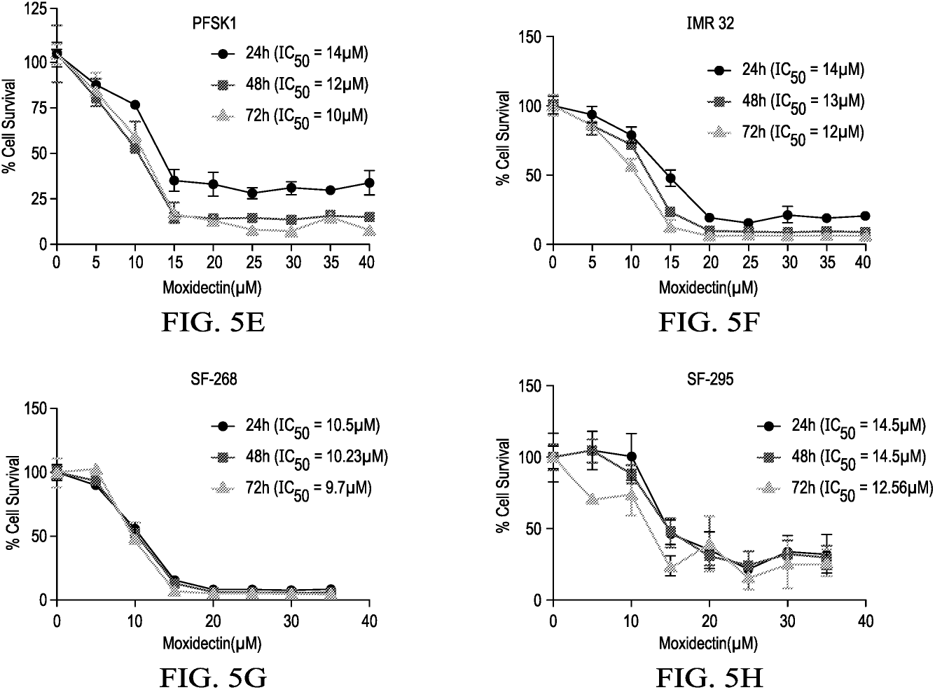

FIGS. 5A-5I shows that Moxidectin treatment suppresses the proliferation of brain cancer cells. Treatment of Daoy (5A), UW228 (5B), ONS76 (5C), UW246 (5D), PFSK1 (medulloblastoma) (5E), IMR32 (5F), SF268 (5G), SF295 (glioblastoma)(5H), and SK-N-AS (neuroblastoma) (5I) cells with varying concentrations of moxidectin displayed significant reduction in the survival of cells in a concentration and time-dependent manner (FIGS. 5A-5I). The IC₅₀ of moxidectin was observed to be 9.7-30 µM in all brain cancer cell lines at 24, 48 and 72-hour time point. These results indicate that moxidectin has the ability to suppress the proliferation of brain cancer cells in a concentration and time-dependent manner.

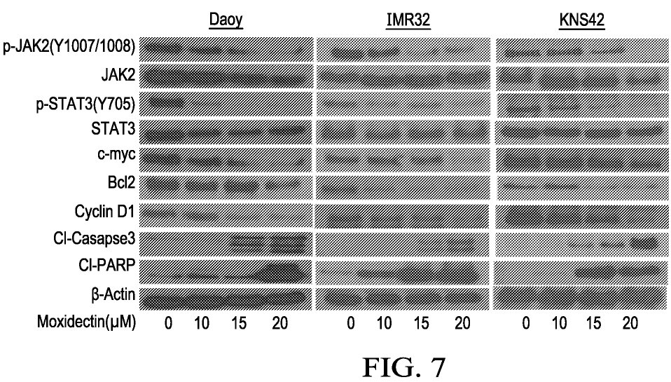

FIGS. 6A-6F show the induction of apoptosis by moxidectin. The mode of cell death caused by moxidectin treatment was determined by Annexin V/FITC assay in Daoy, UW228, ONS76, UW426, PFSK1 and IMR32 brain cancer cells (FIGS. 6A-6F, resepectively). As shown in FIGS. 6A-6F, treatment with moxidectin for 48 hours resulted in a significant increase of apoptosis in all brain cancer cells. Treatment with 10 µM moxidectin had minimal apoptotic effects on all the brain cancer cells tested (FIGS. 6A-6F). 15 µM moxidectin induced -80% apoptosis in Daoy and IMR32 brain cancer cell lines. The percentage of apoptotic cells with 20 µM moxidectin treatment ranged from 90-100% in all the brain cancer cell lines. Induction of apoptosis by moxidectin treatment in these cell lines was further assessed by increase in the cleavage of caspase 3 and PARP (FIG. 7).

FIG. 7 shows that Moxidectin inhibits JAK-STAT signaling: shows that moxidectin mediates its anti-cancer effects in brain cancer cell lines, Daoy, IMR32, and KNS42 cells were treated with 0, 10, 15 and 20 µM moxidectin for 48 hours and evaluated by western blotting. These results show that moxidectin treatment reduced the phosphorylation of Janus Kinase 2 (JAK2) at Tyr1007/1008 in a concentration-dependent manner. However, protein level of JAK2 remained unchanged. It has been reported that, upon activation (phosphorylation) JAK2 activates multiple downstream transcription factors one of which is STAT3. Hyperactivation of STAT3 in cancer cells is strongly attributed to evasion of apoptosis and transcriptional activation of anti-apoptotic proteins such as Bcl-2 and c-myc. These results show that moxidectin treatment reduced the phosphorylation of STAT3 at Tyr705.

Interestingly, it was found that treatment of brain cancer cells with moxidectin resulted in the reduced expression of anti-apoptotic proteins Bcl-2 in Daoy, IMR32 and KNS42 cell lines (FIG. 7). Moreover, moxidectin increased the cleavage of caspase 3 and PARP as an indication of apoptosis, a primary mode of cell death (FIG. 7). These results suggest that the anti-proliferative and apoptosis inducing effects of moxidectin were mediated by inhibiting JAK2 activation and its downstream targets p-STAT3, c-myc, Bcl2 and Cyclin D1.

Moxidectin suppresses the growth of subcutaneously implanted brain cancer cells. To evaluate the ability of moxidectin in inhibiting the growth of brain tumor cells in vivo, ~ 4 million Daoy cells were injected subcutaneously into the right flank of the mice. Once the tumor cells started growing and the tumor volume reached ~ 70 mm³, mice were randomized into four groups. Control group received the vehicle whereas the treatment group received 2.5 mg/kg, 5 mg/kg and 10 mg/kg moxidectin once every day. The tumor readings were taken twice a week throughout the course of the experiment using Vernier caliper. These results showed that 10 mg/kg moxidectin suppressed the growth of subcutaneously injected medulloblastoma cells by 90% (FIG. 4A). Similarly, 2.5 mg/kg and 5 mg/kg moxidectin suppressed Daoy tumor growth by 45% and 60% respectively (FIG. 4C). These results indicate that moxidectin has the potential to inhibit the growth of brain tumor cells in vivo. In another experiment, ~0.3 million Daoy luc cells were injected intracranially in the brain of athymic nude mice. The mice were given 2.5 mg/kg for 33 days. Moxidectin was able to inhibit -65% of intracranially implanted medulloblastoma tumors. These results demonstrate that moxidectin can inhibit medulloblastoma tumor growth in vivo (FIG. 4D). Additionally, ~ 3 million SK-N-AS neuroblastoma cells were injected subcutaneously in the right flank of the mice. Mice were randomized on day 10. Treatment group received 3.5 mg/kg moxidectin. The experiment was terminated on day 23. Moxidectin was able to inhibit 60% of subcutaneously implanted neuroblastoma tumors (FIG. 4D). These results demonstrate that moxidectin can inhibit neuroblastoma progression in vivo.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean ”and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skilled in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### REFERENCES

Cotter DO, Schairer D, Eichenfield L (2018). Emerging therapies for atopic dermatitis: JAK inhibitors. J Am Acad Dermatol: 78(3 Suppl 1 ):S53-S62

Cotreau MM, Warren S. Ryan JL, Fleckenstein L, Vanapalli SR, Brown KR, Rock D, Chen CY, Schwertschlag US (2003). The antiparasitic Moxidectin: Safety, Tolerability and Pharmacokinetics in Humans. Journal of Clinical Pharmacology; 43: 1108-1115

Mitra AK, Agrahari V, Mandal A, Cholkar K, Natarajan C, Shah S, Joseph M, Trinh HM, Vaishya R, Yang X, Hoa Y, Khurana V, Pal D (2015). Novel Delivery approaches for cancer therapeutics. J Control Release; 248-268. doi:1016/j.jconrel.2015.09.067

Mvouaka SK, Menez C, Borin C, Lyazrhi F, Vignault MF, Dupuy J, Collet X, Alvinerie M, Lespine A (2010). Role of P-Glycoprotein in the disposition of macrocyclic lactones: a comparison between ivermectin, eprinomectin, and moxidectin in mice. The American society for pharmacology and experimental therapeutics. DMD 38:573-580

Roskoski R Jr (2016). Janus kinase (JAK) inhibitors in the treatment of inflammatory and neoplastic diseases. Pharmacological Research: 784-803. doi: 10.1016/j.phrs.2016.07.038.

1. A method of inhibiting or reducing deregulated JAK/STAT signaling expression in a subject with a proliferative disease which comprises administering to the subject having or suspected to have the disease, a therapeutically or prophylactically effective amount of the compound of Formula I: 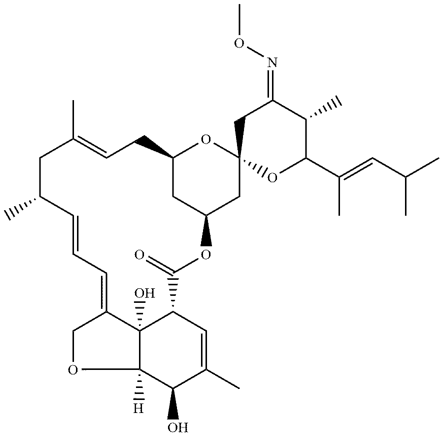
or a pharmaceutically acceptable salt or solvate thereof.

2. The method of claim 1, wherein the disease is selected from at least one brain, medulloblastoma, neuroblastoma, glioblastoma, leukemia, myeloma, myeloproliferative disease, myelodysplastic syndrome, idiopathic hypereosinophilic syndrome (HES), bladder cancer, breast cancer, cervical cancer, CNS cancer, colon cancer, esophageal cancer, head and neck cancer, liver cancer, lung cancer, nasopharyngeal cancer, neuroendocrine cancer, ovarian cancer, pancreatic cancer, prostate cancer, renal cancer, salivary gland cancer, small cell lung cancer, skin cancer, stomach cancer, testicular cancer, thyroid cancer, uterine cancer, and hematologic malignancy, or involves dysregulation of JAK/STAT signaling selected from at least one of: Polycythemia vera (PV), idiopathic hypereosinophilic syndrome (HES), Severe combined immunodeficiency (SCID), Hyperimmunoglobulin E syndrome (HIES, or Job’s syndrome), Mucocutaneous candidiasis, Mycobacterial infection, and autoimmune disorders, and optionally the disease is relapsed/refractory to a prior JAK kinase inhibitor.

3. (canceled)

4. The method of claim 1, wherein at least one of:
the therapeutically or prophylactically effective amounts are from about 0.1 to 25, 1 to 15, 2 to 10, 3 to 8, 5 to 15, 2, 3, 4, 5, 6, 7, 8, 9, 10, 12, 15, 20, 25, 30, 35, 40, 45, 50, 55, 60, 65, 70, 75, 80, 85, 90, or 100 mg per day; or
is provided at 2.5 mg/kg, 5 mg/kg, or 10 mg/kg moxidectin once every day.

5. The method of claim 1, wherein the compound is administered at least one of continuously, intermittently, systemically, or locally.

6. The method of claim 1, wherein deregulated JAK kinase is defined further as a mutated JAK kinase that is constitutively active.

7. The method of claim 1, wherein the compound is administered orally, intravenously, or intraperitoneally.

8. The method of claim 1, wherein the compound reduces expression or activity of at least one of: p-STAT3, STAT3, c-myc, Bcl2, or Cyclin D1.

9. The method of claim 1, wherein at least one of:
the therapeutically or prophylactically effective amount of the compound is administered daily for as long as the subject is in need of treatment for the proliferative disease,
the compound is provided at least one of sequentially or concomitantly, with another pharmaceutical agent in a newly diagnosed proliferative disease subject, to maintain remission of an existing subject or a relapsed/refractory proliferative disease subject,
the compound is provided as a single agent or in combination with another pharmaceutical agent in a newly diagnosed proliferative disease subject, to maintain remission, or a relapsed/refractory proliferative disease subject; or
the compound is provided as a single agent or in combination with another pharmaceutical agent in a newly diagnosed proliferative disease pediatric subject to maintain remission, or a relapsed/refractory proliferative disease pediatric.

10. (canceled)

11. (canceled)

12. (canceled)

13. (canceled)

14. (canceled)

15. A method for treating a subject with a proliferative disease comprising:
administering to the subject in need of such treatment a therapeutically effective amount of Moxidectin or a salt thereof, wherein the disorder is characterized by deregulated JAK/STAT signaling activity, disease is selected from at least one of a brain, medulloblastoma, neuroblastoma, glioblastoma, and optionally is refractory to at least one other tyrosine kinase inhibitor.

16. The method of claim 15, wherein the compound is administered orally, intravenously, or intraperitoneally, topically, transdermally, vaginally, sublingually.

17. The method of claim 15, wherein the compound reduces expression or activity of at least one of: p-STAT3, c-myc, Bcl2, or Cyclin D1.

18. The method of claim 15, wherein at least one of:
the Moxidectin is provided at least one of sequentially or concomitantly, with chemotherapy, radiotherapy, or surgery in a newly diagnosed proliferative disease, to maintain remission, or a relapsed/refractory proliferative disease,
the Moxidectin is provided as a single agent or in combination with chemotherapy, radiotherapy, immunotherapy or surgery for treatment of pediatric subject with the proliferative disease,
the Moxidectin is provided as a single agent to at least one of post standard induction therapy, or high dose induction therapy, in newly diagnosed proliferative disease; or
the Moxidectin is provided as a single agent in treatment of subjects with the proliferative disease that is either refractory to, or has relapsed after, standard or high dose chemotherapy, radiotherapy, immunotherapy, or surgery.

19. (canceled)

20. (canceled)

21. (canceled)

22. (canceled)

23. (canceled)

24. The method of claim 15, wherein the compound is provided at 2.5 mg/kg, 5 mg/kg,or 10 mg/kg moxidectin once every day, or
is provided at 0.1 to 25, 1 to 15, 2 to 10, 3 to 8, 5 to 15, 2, 3, 4, 5, 6, 7, 8, 9, 10, 12, 15, 20, 25, 30, 35, 40, 45, 50, 55, 60, 65, 70, 75, 80, 85, 90, or 100 mg per day.

25. A method for treating a subject with brain cancer comprising:
obtaining a sample from the subject suspected of having a brain cancer;
determining from the subject sample that the subject has a deregulated JAK receptor tyrosine kinase; and
administering to the subject in need of such treatment a therapeutically effective amount of Moxidectin or a salt thereof, wherein the brain cancer is characterized by deregulated JAK receptor tyrosine kinase activity.

26. A method for specifically inhibiting a deregulated receptor tyrosine kinase comprising:
obtaining a subject sample and determining which receptor tyrosine kinases are deregulated; and
administering to a mammal in need of such treatment a therapeutically effective amount of Moxidectin or a salt thereof, wherein the deregulated receptor tyrosine kinase is a JAK receptor tyrosine kinase.

27. The method of claim 25, wherein the deregulated receptor tyrosine kinase is in a cancer selected from at least one of a brain, medulloblastoma, neuroblastoma, or glioblastoma, and optionally is relapsed/refractory to a prior JAK tyrosine kinase inhibitor, and optionally the deregulated JAK2 is defined further as a mutated JAK2 is constitutively active.

28. The method of claim 25, wherein the therapeutically and prophylactically effective amounts are from about 0.1 to 25, 1 to 15, 2 to 10, 3 to 8, 5 to 15, 2, 3, 4, 5, 6, 7, 8, 9, 10, 12, 15, 20, 25, 30, 35, 40, 45, 50, 55, 60, 65, 70, 75, 80, 85, 90, or 100 mg per day; or
is provided at 2.5 mg/kg, 5 mg/kg, or 10 mg/kg moxidectin once every day.

29. The method of claim 25, wherein the compound is administered at least one of continuously, intermittently, systemically, or locally.

30. (canceled)

31. The method of claim 25, wherein the compound is administered orally, intravenously, or intraperitoneally.

32. The method of claim 25, wherein the compound reduces expression of antiapoptotic protein Bcl-2.

33. The method of claim 25, wherein at least one of:
the therapeutically or prophylactically effective amount of the compound is administered daily for as long as the subject is in need of treatment for the proliferative disease,
the subject is provided treatment and the method further comprises the steps of: obtaining one or more subject samples to determine the effect of the treatment and continuing treatment until the proliferative disease is reduced or eliminated;
the compound is provided at least one of sequentially or concomitantly, with another pharmaceutical agent in a newly diagnosed proliferative disease subject, to maintain remission, or a relapsed/refractoryproliferative disease subject;
the compound is provided as a single agent or in combination with another pharmaceutical agent in a newly diagnosed proliferative disease subject, to maintain remission, or a relapsed/refractory proliferative disease subject; or
the compound is provided as a single agent or in combination with another pharmaceutical agent in a newly diagnosed proliferative disease pediatric subject, to maintain remission, or a relapsed/refractory proliferative disease pediatric subject.

34. (canceled)

35. (canceled)

36. (canceled)

37. (canceled)

38. (canceled)

39. A method for treating a subject with cancer comprising: obtaining a sample suspected of having cancer from the subject;
determining if the subject has a cancer become resistant to prior JAK protein tyrosine kinase inhibition; and
administering a therapeutically effective amount of Moxidectin or a salt thereof to overcome the resistance to the prior JAK protein tyrosine kinase inhibition.
